(19) United States
(12) Patent Application Publication
(10) Pub. No.: US 2026/0029375 A1
(43) Pub. Date: Jan. 29, 2026

(54) METHODS OF AND APPARATUSES FOR INSPECTING AN OBJECT

(71) Applicant: KAL TIRE, Vernon (CA)

(72) Inventors: Clayton MCMUNN, Vernon (CA); Kurt RUFFINI, Vernon (CA); Jan Peter NILSSON, Vernon (CA)

(73) Assignee: KAL TIRE, Vernon, BC (CA)

(21) Appl. No.: 19/102,469

(22) PCT Filed: Sep. 2, 2022
(86) PCT No.: PCT/CA2022/051329

§ 371 Date: Feb. 10, 2025

Related U.S. Application Data
(60) Provisional application No. 63/397,763, filed on Aug. 12, 2022.
(60) Provisional application No. 63/397,784, filed on Aug. 12, 2022.

(51) Int. Cl. G01N 27/9013; G01N 27/904; G01N 27/9093; G01N 33/00

(52) U.S. Cl. CPC G01N 27/902; G01N 27/904; G01N 27/9093; G01N 33/0085

(57) ABSTRACT

A method of inspecting an object includes: moving a support body relative to the object in a first direction along a surface of the object; wherein the support body supports at least one sensor such that moving the support body relative to the object in the first direction causes the at least one sensor to move relative to the object in the first direction; and wherein the at least one sensor is movable relative to the support body in a second direction along the surface of the object and different from the first direction during at least a portion of time while moving the support body relative to the object in the first direction. Apparatuses are also disclosed.

### RELATED APPLICATIONS

This application claims the benefit of, and priority to, U.S. provisional patent application Nos. 63/397,763 and 63/397,784, both filed Aug. 12, 2022. The entire contents of U.S. provisional patent application Nos. 63/397,763 and 63/397,784 are incorporated by reference herein.

### FIELD

This disclosure relates generally to inspecting one or more objects.

### RELATED ART

Objects, such as wheels for off-the-road (“OTR”) vehicles or other wheels for example, may be inspected, for example as part of a process to detect possible defects such as corrosion, wear, or other damage or imperfections that may arise over time. Non-destructive testing (“NDT”) techniques can be used to inspect objects, but skilled NDT technicians can be costly and may not be available-often enough, or at all-at locations (such as remote mining sites, for example) where the inspection of objects may be required. Therefore, sometimes wheels or other objects must be transported long distances to locations where NDT is available. Further, some objects, such as wheels for OTR vehicles for example, may be large, and NDT of such objects may require time-consuming steps (such as washing and removing paint before inspection, and repainting after inspection), so NDT of some objects can be time-consuming and costly.

Alternatively, wheels or other objects may simply be discarded and replaced, for example after a threshold number of hours of use, which can be wasteful because objects may be discarded and replaced when the objects may still be in an acceptable condition or may be capable of being repaired.

### SUMMARY

According to at least one embodiment, there is disclosed a method of inspecting an object, the method comprising: moving a support body relative to the object in a first direction along a surface of the object; wherein the support body supports at least one sensor such that moving the support body relative to the object in the first direction causes the at least one sensor to move relative to the object in the first direction; and wherein the at least one sensor is movable relative to the support body in a second direction along the surface of the object and different from the first direction during at least a portion of time while moving the support body relative to the object in the first direction.

In at least some embodiments, during at least a portion of time while moving the support body relative to the object in the first direction, the support body is supported by the object.

In at least some embodiments, the method further comprises positioning a support surface of the support body on the object such that the object supports the support body.

In at least some embodiments, a sensor-support body supports the at least one sensor relative to the support body.

In at least some embodiments, the sensor-support body permits movement of the at least one sensor relative to the support body in the second direction.

In at least some embodiments, at least a portion of the sensor-support body is rotatable relative to the support body, and the sensor-support body permits the movement of the at least one sensor relative to the support body in the second direction by rotation of the at least a portion of the sensor-support body relative to the support body.

In at least some embodiments, the sensor-support body permits movement of the at least one sensor relative to the support body in a third direction different from the first and second directions and towards and away from the support body.

In at least some embodiments, the method further comprises adjusting a position of the sensor-support body relative to the support body.

In at least some embodiments, adjusting the position of the sensor-support body relative to the support body comprises adjusting a position of the sensor-support body along an elongate body of the support body.

In at least some embodiments, a first at least one resilient body resiliently urges the at least one sensor against the surface of the object.

In at least some embodiments, the method further comprises adjusting a resilient force urged by the first at least one resilient body on the at least one sensor.

In at least some embodiments, adjusting the resilient force urged by the first at least one resilient body on the at least one sensor comprises adjusting a location, relative to the support body, where a first portion of the first at least one resilient body is attached to the support body, and a second portion of the first at least one resilient body exerts the resilient force on the at least one sensor.

In at least some embodiments, the first at least one resilient body resiliently urges the at least one sensor against the surface of the object in the third direction.

In at least some embodiments, movement of the at least one sensor relative to the support body in the second direction is against a resilient force from a second at least one resilient body.

In at least some embodiments, the second at least one resilient body urges the at least one sensor in the second direction towards an adjustable default position of the at least one sensor relative to the support body.

In at least some embodiments, the adjustable default position is one of a plurality of discrete adjustable default positions of the at least one sensor relative to the support body.

In at least some embodiments, moving the support body relative to the object in the first direction comprises causing at least one motor to move the support body relative to the object in the first direction.

In at least some embodiments, the support body further supports at least one computing device in communication with the at least one sensor.

In at least some embodiments, the at least one computing device controls the at least one motor to move the support body relative to the object in the first direction.

In at least some embodiments, the computing device controls the at least one motor to move the support body at a generally constant speed relative to the object.

In at least some embodiments, the object is generally cylindrical.

In at least some embodiments, the first direction is a peripheral direction around the object.

In at least some embodiments, the second direction is an axial direction along the object.

In at least some embodiments, the third direction is generally radial relative to the object.

In at least some embodiments, the object comprises a wheel.

In at least some embodiments, the object comprises a wheel of a mining vehicle.

In at least some embodiments, the object comprises an off-the-road (OTR) wheel.

In at least some embodiments, the surface comprises an outer surface of the object.

In at least some embodiments, the surface comprises an inner surface of the object.

In at least some embodiments, the at least one sensor comprises at least one eddy-current probe array.

In at least some embodiments, the at least one sensor comprises at least one sensor of movement of the support body relative to the object.

According to at least one embodiment, there is disclosed an apparatus for inspecting an object, the apparatus comprising: a support body movable relative to the object in a first direction along a surface of the object; wherein the support body is configured to support at least one sensor such that moving the support body relative to the object in the first direction causes the at least one sensor to move relative to the object in the first direction, and such that the at least one sensor is movable relative to the support body in a second direction along the surface of the object and different from the first direction while moving the support body relative to the object in the first direction.

In at least some embodiments, the support body defines a space for receiving at least a portion of the object, the support body comprising at least one support surface facing the space to support the support body on the object when the space receives the at least a portion of the object.

In at least some embodiments, the apparatus further comprises a sensor-support body configured to support the at least one sensor relative to the support body.

In at least some embodiments, the sensor-support body permits movement of the at least one sensor relative to the support body in the second direction.

In at least some embodiments, at least a portion of the sensor-support body is rotatable relative to the support body, and the sensor-support body permits the movement of the at least one sensor relative to the support body in the second direction by rotation of the at least a portion of the sensor-support body relative to the support body.

In at least some embodiments, the sensor-support body permits movement of the at least one sensor relative to the support body in a third direction different from the first and second directions and towards and away from the support body.

In at least some embodiments, a position of the sensor-support body relative to the support body is adjustable.

In at least some embodiments, a position of the sensor-support body along an elongate body of the support body is adjustable.

In at least some embodiments, the apparatus further comprises a first at least one resilient body configured to urge resiliently the at least one sensor against the surface of the object.

In at least some embodiments, a resilient force urged by the first at least one resilient body on the at least one sensor is adjustable.

In at least some embodiments, the apparatus further comprises a resilient-body-attachment body having an adjustable position relative to the support body, and a first portion of the first at least one resilient body is attached to the resilient-body-attachment body and a second portion of the first at least one resilient body exerts the resilient force on the at least one sensor such that adjusting the adjustable position of the resilient-body-attachment body relative to the support body adjusts the resilient force urged by the first at least one resilient body on the at least one sensor.

In at least some embodiments, the first at least one resilient body is configured to urge resiliently the at least one sensor against the surface of the object in the third direction.

In at least some embodiments, the apparatus further comprises a second at least one resilient body configured to impose a resilient force against movement of the at least one sensor relative to the support body in the second direction.

In at least some embodiments, the second at least one resilient body is configured to urge the at least one sensor in the second direction towards an adjustable default position of the at least one sensor relative to the support body.

In at least some embodiments, the adjustable default position is one of a plurality of discrete adjustable default positions of the at least one sensor relative to the support body.

In at least some embodiments, the apparatus further comprises at least one motor configured to move the support body relative to the object in the first direction.

In at least some embodiments, the apparatus further comprises at least one computing device in communication with the at least one sensor, and the support body further supports the at least one computing device.

In at least some embodiments, the at least one computing device is configured to control the at least one motor to move the support body relative to the object in the first direction.

In at least some embodiments, the computing device is configured to control the at least one motor to move the support body relative to the object in the first direction at a generally constant speed relative to the object.

In at least some embodiments, the support body is configured to be supported by the object such that the support body is movable relative to the object in the first direction.

In at least some embodiments, the object is generally cylindrical.

In at least some embodiments, the first direction is a peripheral direction around the object.

In at least some embodiments, the second direction is an axial direction along the object.

In at least some embodiments, the third direction is generally radial relative to the object.

In at least some embodiments, the object comprises a wheel.

In at least some embodiments, the object comprises a wheel of a mining vehicle.

In at least some embodiments, the object comprises an off-the-road (OTR) wheel.

In at least some embodiments, the surface comprises an outer surface of the object.

In at least some embodiments, the surface comprises an inner surface of the object.

In at least some embodiments, the apparatus further comprises the at least one sensor.

In at least some embodiments, the at least one sensor comprises at least one eddy-current probe array.

In at least some embodiments, the at least one sensor comprises at least one sensor of movement of the support body relative to the object.

According to at least one embodiment, there is disclosed a system comprising the apparatus and the object.

According to at least one embodiment, there is disclosed use of the apparatus for inspecting the object.

Other aspects and features will become apparent to those ordinarily skilled in the art upon review of the following description of illustrative embodiments in conjunction with the accompanying figures.

### BRIEF DESCRIPTION OF THE DRAWINGS

FIG. 1 is a perspective view of an inspection system according to one embodiment.

FIG. 2 is another perspective view of the system of FIG. 1.

FIG. 3 is a perspective view of a sensor-support body of the system of FIG. 1.

FIG. 4 is an exploded perspective view of the sensor-support body of FIG. 3.

FIGS. 5A and 5B include details of parts of the sensor-support body of FIG. 3.

FIGS. 6A and 6B are front perspective views of a pivot body of the sensor-support body of FIG. 3.

FIG. 6C is a front view of the pivot body of FIGS. 6A and 6B.

FIG. 6D is a side elevation view of the pivot body of FIGS. 6A and 6B.

FIG. 6E is a top plan view of the pivot body of FIGS. 6A and 6B.

FIG. 6F is a cross-sectional view of the pivot body of FIGS. 6A and 6B, taken along the line 6F-6F in FIG. 6C.

FIG. 6G is a fragmentary view of a portion of the pivot body of FIGS. 6A and 6B, as shown in FIG. 6D.

FIG. 7A is a front perspective view of a slider body of the sensor-support body of FIG. 3.

FIG. 7B is a rear perspective view of the slider body of FIG. 7A.

FIG. 7C is a top plan view of the slider body of FIG. 7A.

FIG. 7D is a right-side elevation view of the slider body of FIG. 7A.

FIG. 7E is a rear view of the slider body of FIG. 7A.

FIG. 7F is a left-side elevation view of the slider body of FIG. 7A.

FIG. 7G is a front view of the slider body of FIG. 7A.

FIG. 7H is a fragmentary view of a portion of the slider body of FIG. 7A, as shown in FIG. 7D.

FIG. 7I is a fragmentary view of a portion of the slider body of FIG. 7A, as shown in FIG. 7G.

FIG. 7J is a cross-sectional view of the slider body of FIG. 7A, taken along the line 7J-7J in FIG. 7E.

FIG. 8A is a front perspective view of a guide plate of the sensor-support body of FIG. 3.

FIG. 8B is a rear perspective view of the guide plate of FIG. 8A.

FIG. 8C is a top plan view of the guide plate of FIG. 8A.

FIG. 8D is a front view of the guide plate of FIG. 8A.

FIG. 8E is a rear view of the guide plate of FIG. 8A.

FIG. 8F is a side elevation view of the guide plate of FIG. 8A.

FIG. 8G is a fragmentary view of a portion of the guide plate of FIG. 8A, as shown in FIG. 8F.

FIG. 9A is a perspective view of a clevis of the sensor-support body of FIG. 3.

FIG. 9B is a top plan view of the clevis of FIG. 9A.

FIG. 9C is a left-side elevation view of the clevis of FIG. 9A.

FIG. 9D is a front view of the clevis of FIG. 9A.

FIG. 9E is a right-side elevation view of the clevis of FIG. 9A.

FIG. 10A is a perspective view of a spring hook of the sensor-support body of FIG. 3.

FIG. 10B is a right-side elevation view of the spring hook of FIG. 10A.

FIG. 10C is a left-side elevation view of the spring hook of FIG. 10A.

FIG. 10D is a top plan view of the spring hook of FIG. 10A.

FIG. 10E is a rear view of the spring hook of FIG. 10A.

FIG. 11A is a perspective view of a mounting body of the sensor-support body of FIG. 3.

FIG. 11B is a front view of the mounting body of FIG. 11A.

FIG. 11C is a top plan view of the mounting body of FIG. 11A.

FIG. 11D is a right-side elevation view of the mounting body of FIG. 11A.

FIG. 12A is a perspective view of a spring shaft of the sensor-support body of FIG. 3.

FIG. 12B is a left-side elevation view of the spring shaft of FIG. 12A.

FIG. 12C is a front view of the spring shaft of FIG. 12A.

FIG. 13 is a top plan view of the sensor-support body of FIG. 3.

FIG. 14 is an isometric view of the sensor-support body of FIG. 3.

FIG. 15 is a front view of the sensor-support body of FIG. 3.

FIG. 16 is a right-side elevation view of the sensor-support body of FIG. 3.

FIG. 17 illustrates rotation of the pivot body of FIGS. 6A and 6B relative to the mounting body of FIG. 11A.

FIG. 18 illustrates rotation of the slider body of FIG. 7A relative to the pivot body of FIGS. 6A and 6B.

FIG. 19 is a perspective view of a portion of the system of FIG. 1.

FIG. 20 illustrates linear movement of the guide plate of FIG. 8A and of a guide rail of the sensor-support body of FIG. 3 relative to the slider body of FIG. 7A.

FIGS. 21-25 are prespective views of portions of the system of FIG. 1, illustrating examples of operation of the system of FIG. 1.

### DETAILED DESCRIPTION

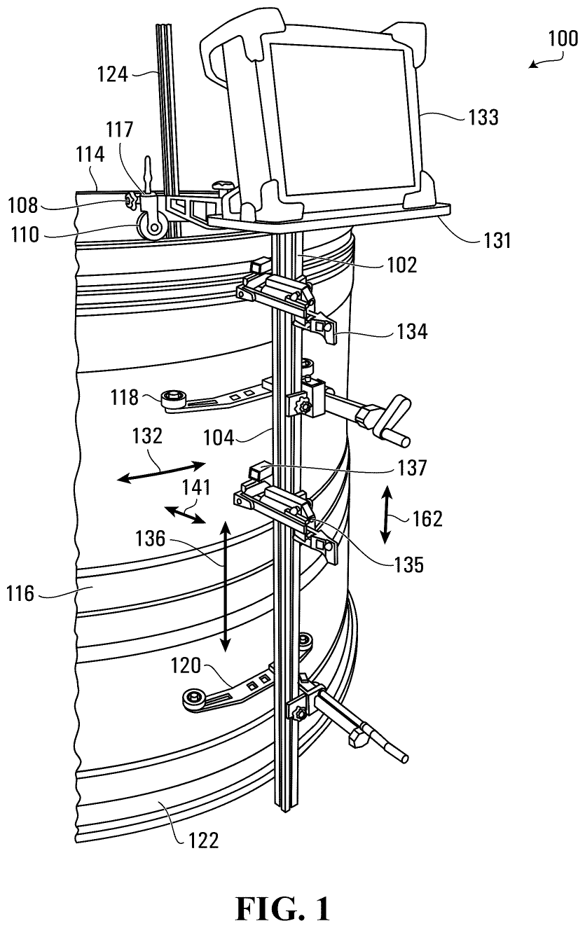

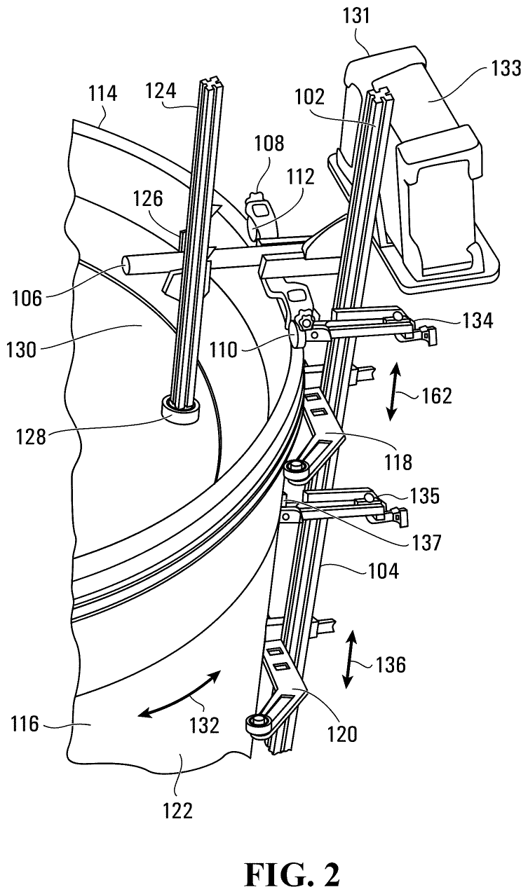

Referring to FIGS. 1 and 2, an inspection system according to one embodiment is shown generally at 100 and includes a support body 102. The support body 102 includes a first elongate portion 104 and a second elongate portion 106 coupled to the first elongate portion 104. A slidable support body 108 is coupled to the second elongate portion 106 and includes wheels 110 and 112. Outer surfaces of the wheels 110 and 112 are positioned to contact a surface of an object, such as a surface (or end surface) of an inner rim 114 of a wheel 116 for an off-the-road (OTR) vehicle, to facilitate the slidable support body 108 (and thus the support body 102) sliding relative to the wheel 116. As a result, the support body 102 is supported by the wheel 116, which is an example of an object, and the outer surfaces of the wheels 110 and 112 are examples of support surfaces. In the embodiment shown, the slidable support body 108 includes at least one motor 117 operable to turn one or both of the wheels 110 and 112.

However, alternative embodiments may differ. For example, alternative embodiments may include one or more alternatives to the slidable support body 108 that may or may not include wheels. Also, the wheel 116 is a generally cylindrical wheel for an OTR vehicle, but an alternative embodiment may include one or more other objects that may include other wheels and that may be generally cylindrical, such as a different wheel for an OTR vehicle, a wheel for a mining vehicle, or a wheel for another vehicle. In this context, “generally cylindrical” refers to a wheel that may not be perfectly cylindrical, but that may function substantially similarly to a cylindrical wheel. More generally, “generally” herein contemplates variations that may or may not be described herein and that may function substantially similar to those described herein. Further, an alternative embodiment may include one or more other objects that may or may not include wheels. In some embodiments, such objects may be partly or entirely ferromagnetic.

The first elongate portion 104 is also coupled to slidable bodies 118 and 120, each of which may be similar to the slidable support body 108 but may be positioned to contact and slide against an outer surface 122 of the wheel 116 when the outer surfaces of the wheels 110 and 112 contact the surface of the inner rim 114. The slidable bodies 118 and 120 may be adjustable (for example using cranks shown in FIGS. 1 and 2) to adjust positions of the slidable bodies 118 and 120 relative to the first elongate portion 104, for example to position the slidable bodies 118 and 120 in contact with the outer surface 122 or to adjust a separation distance between the first elongate portion 104 and the outer surface 122.

The support body 102 also includes a third elongate portion 124 coupled to the second elongate portion 106 using a clamp 126 that allows the third elongate portion 124 to be coupled to the second elongate portion 106 at different adjustable positions along the second elongate portion 106. A slidable body 128 is coupled to the third elongate portion 124 and may be positioned to contact and slide against an inner surface 130 of the wheel 116 opposite the outer surface 122 when the outer surfaces of the wheels 110 and 112 contact the surface of the inner rim 114 and when the slidable bodies 118 and 120 contact and slide against the outer surface 122. The surface of the inner rim 114 is between the outer surface 122 and the inner surface 130.

As shown in FIGS. 1 and 2, the support body 102 defines a space (between the elongate portions 104, 106, and 124) to receive at least a portion of the wheel 116, and the outer surfaces of the wheels 110 and 112 face into the space to support the support body 102 on the wheel 116 when the space receives at least a portion of the wheel 116.

As a result, the support body 102 is configured to be supported by the wheel 116, and when the wheel 116 supports the support body 102, the support body 102 is movable relative to the wheel 116 in a direction (or first direction) 132 along the outer surface 122. In the embodiment shown, the wheel 116 is generally cylindrical, and the direction 132 is a peripheral or rotational direction generally around an axis of rotation of the wheel 116, or a generally lateral direction in the orientation of FIGS. 1 and 2. Herein, a “peripheral” or a “rotational” direction is not limited to a geometrically precise peripheral or rotational direction, but rather may include directions that are substantially similar to a peripheral or rotational direction.

In some embodiments, the at least one motor 117 may turn one or both of the wheels 110 and 112 to cause, control, or both cause and control movement of the support body 102 relative to the wheel 116 in the direction 132. In general, such motorization may facilitate inspection of an object such as the wheel 116. Further, such motorization may cause the support body 102 to move relative to the wheel 116 in the direction 132 at a constant or generally constant speed, which may facilitate inspection of an object such as the wheel 116.

However, alternative embodiments may differ. For example, in alternative embodiments, a support body may be configured to be supported by a wheel or by another object in other ways. Also, in alternative embodiments, objects and directions as described above may differ, and motorization may differ or may be omitted in alternative embodiments. For example, in some embodiments, the support body 102 may be moved, relative to the wheel 116 in the direction 132, manually or by one or more other alternatives to the at least one motor 117.

The support body 102 also includes a support structure 131 for supporting a computing device 133. The computing device 133 may receive one or more signals from one or more sensors (or probes) as described herein to facilitate inspection of an object such as the wheel 116. Further, the computing device 133 may control the at least one motor 117 to cause, control, or both cause and control movement of the support body 102 relative to the wheel 116 in the direction 132.

The support body 102 is an example only, and alternatives may differ. For example, alternative embodiments may include other structures that may differ from the elongate portions 104, 106, and 124. Further, alternative embodiments may include one or more alternatives to the slidable bodies 118, 120, and 128, or otherwise may include fewer, more, or different parts.

Referring to FIGS. 1-3, the system 100 also includes a sensor-support body 134 and a sensor-support body 135. The sensor-support body 134 is described below, and in the embodiment shown, the sensor-support body 135 is similar to the sensor-support body 134, although alternative embodiments may differ. The sensor-support body 135, and thus the support body 102, may support a sensor (or encoder) 137. In the embodiment shown, the sensor 137 senses movement of the sensor 137, and thus of the support body 102, relative to the wheel 116. For example, the sensor 137 may include one or more wheels that are may be positioned to contact the outer surface 122 such that, when the one or more wheels contact the outer surface 122, movement of the sensor 137 (and thus of the support body 102) relative to the wheel 116 may be measured by measuring rotation of the one or more wheels. As another example, the sensor 137 may include one or more optical sensors that are operable to sense movement of the sensor 137 relative to the wheel 116. However, alternative embodiments may include more or fewer sensor-support bodies that may differ from the sensor-support bodies described herein. Further, the sensor 137 is an example only, and alternative embodiments may differ.

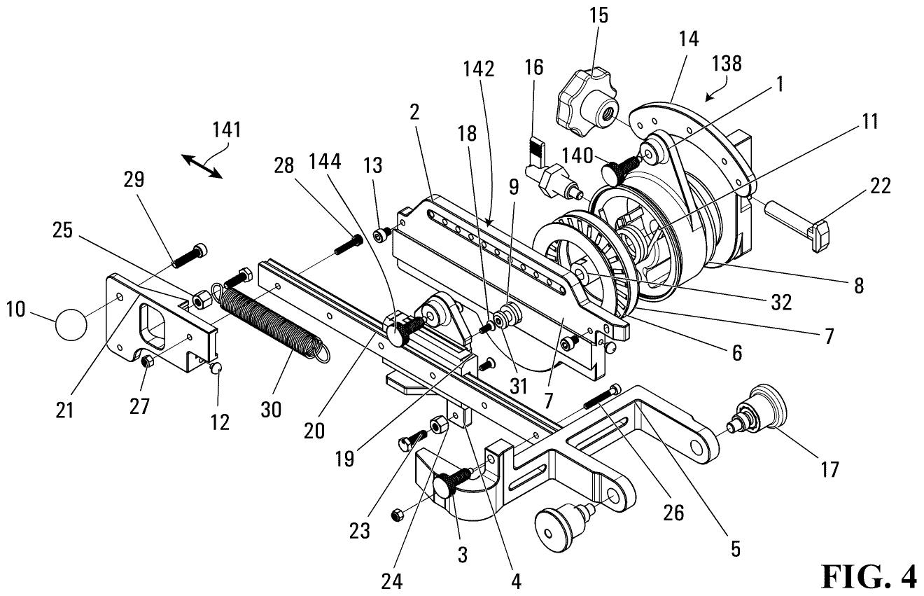

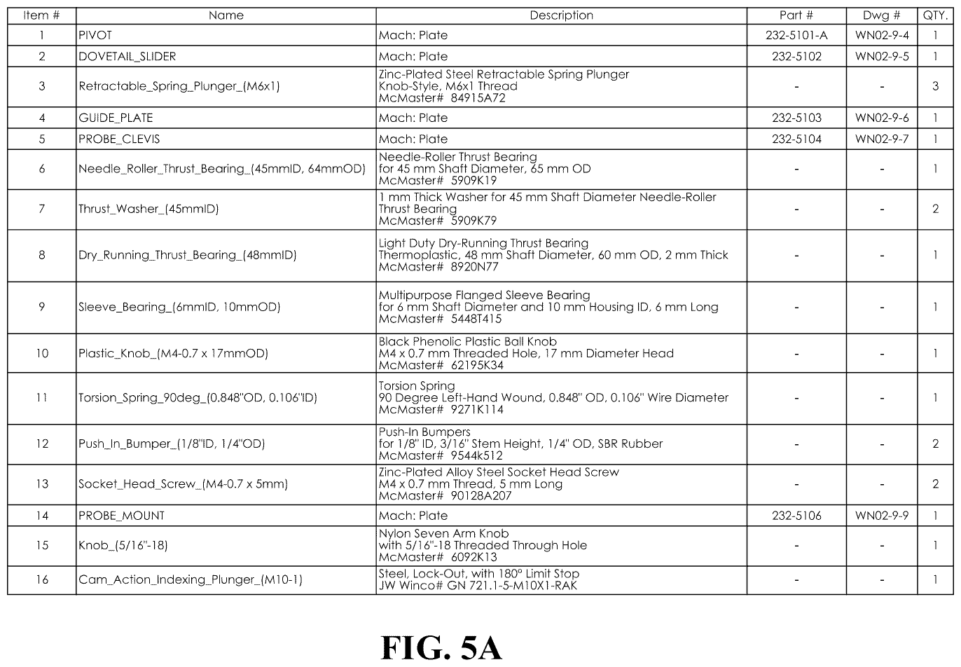

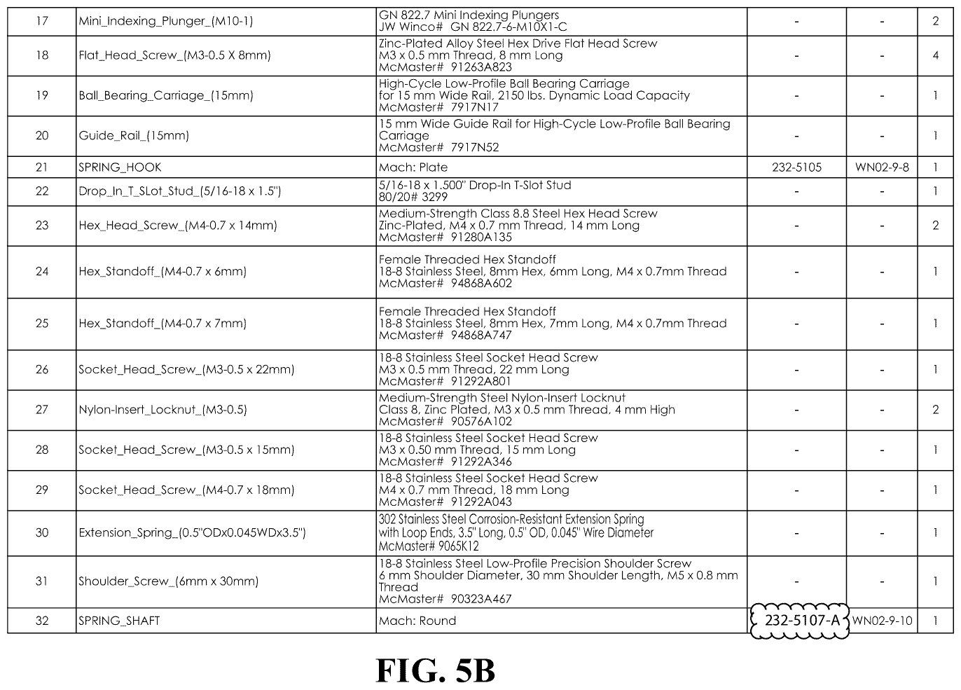

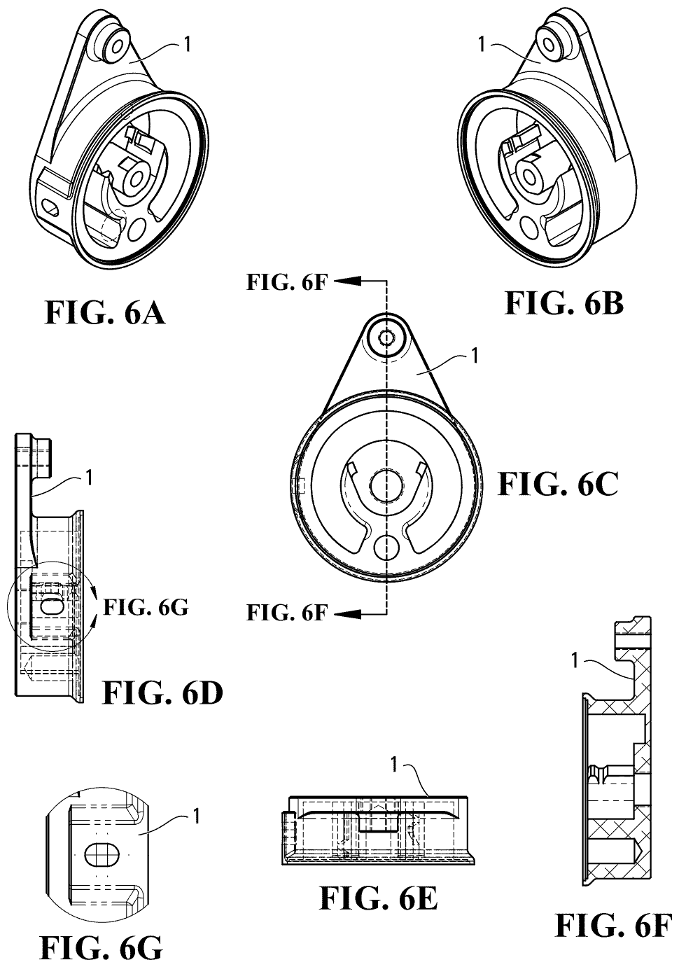

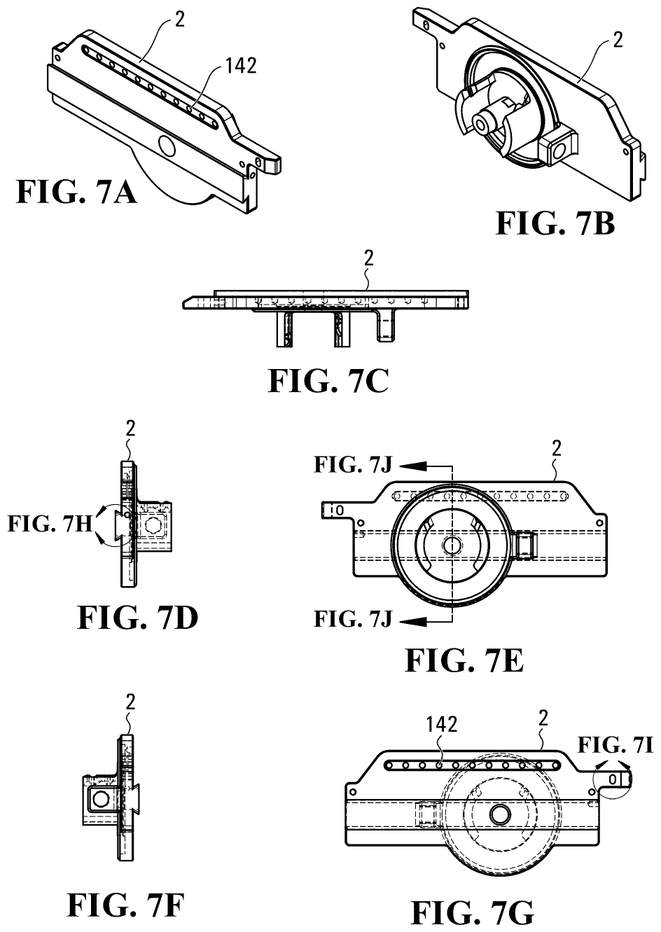

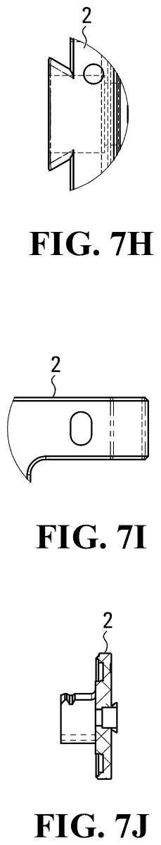

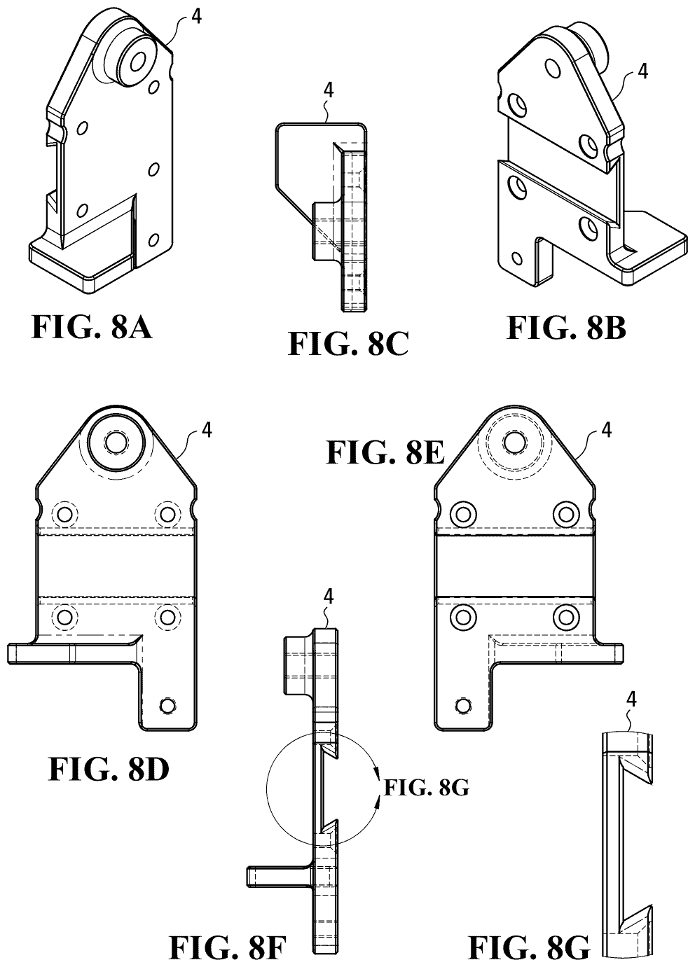

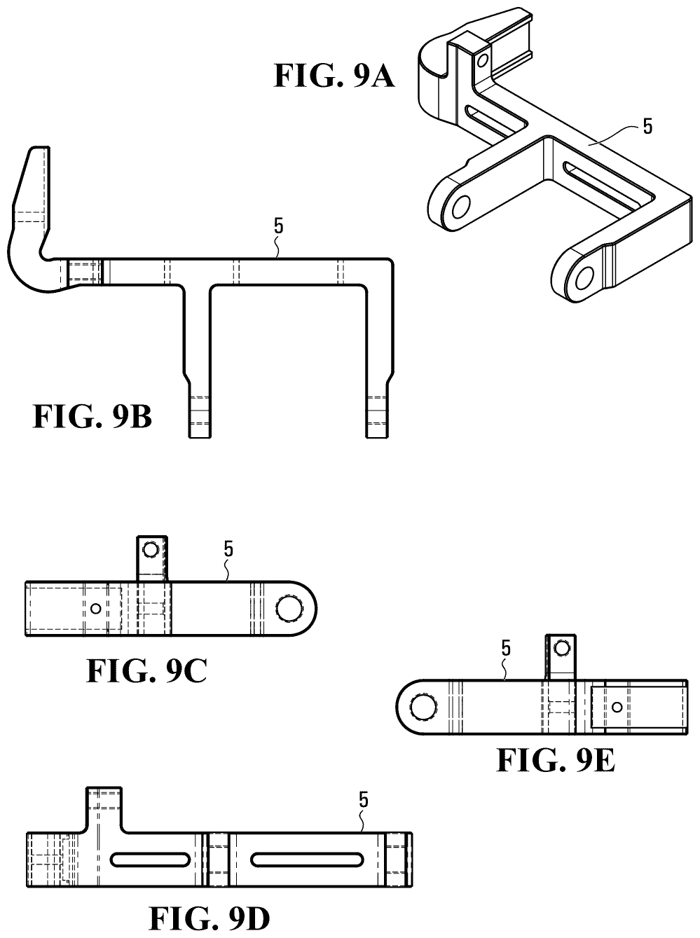

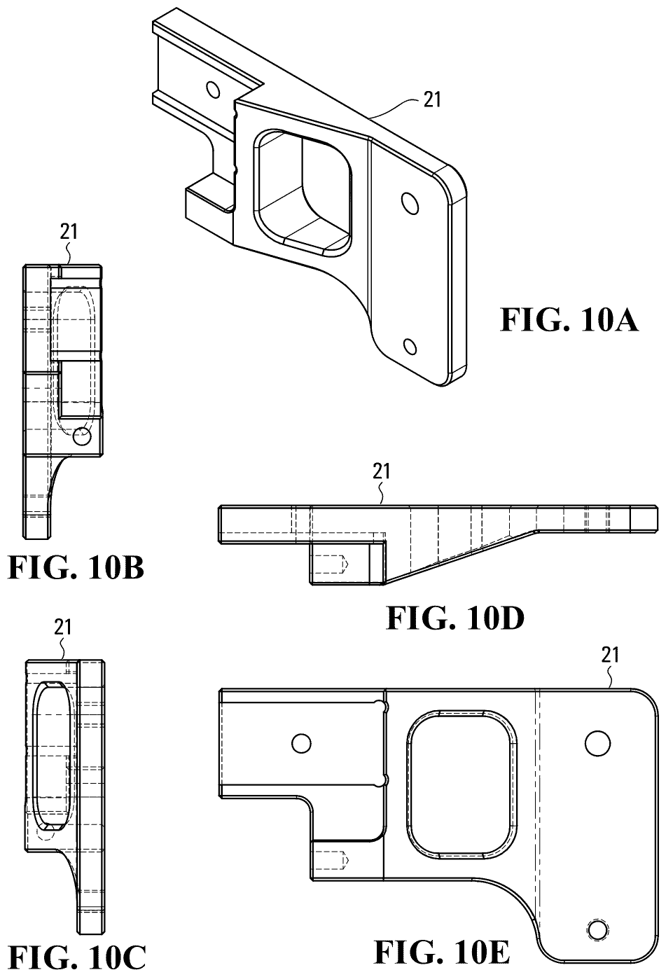

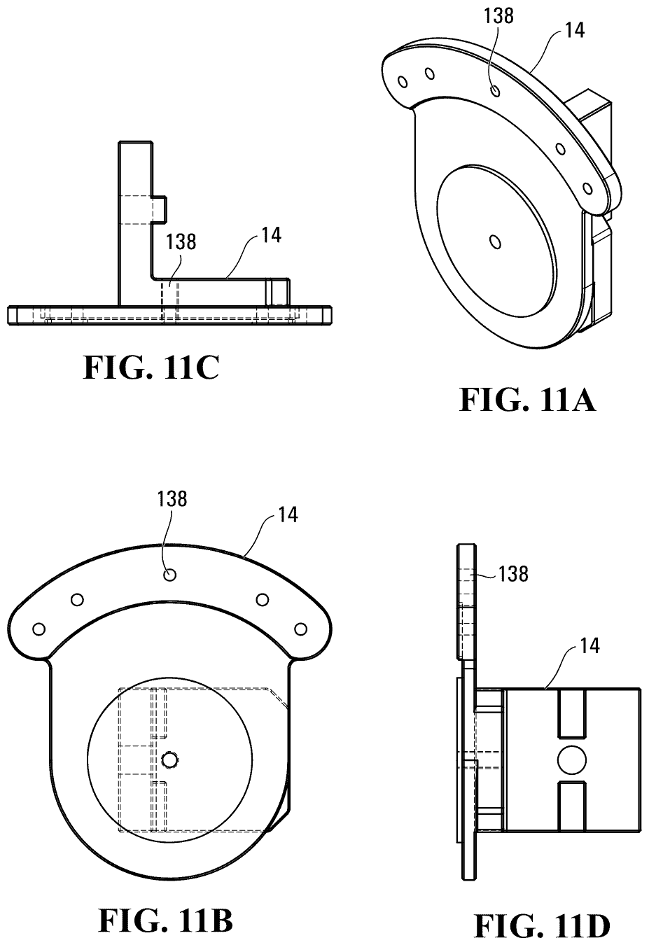

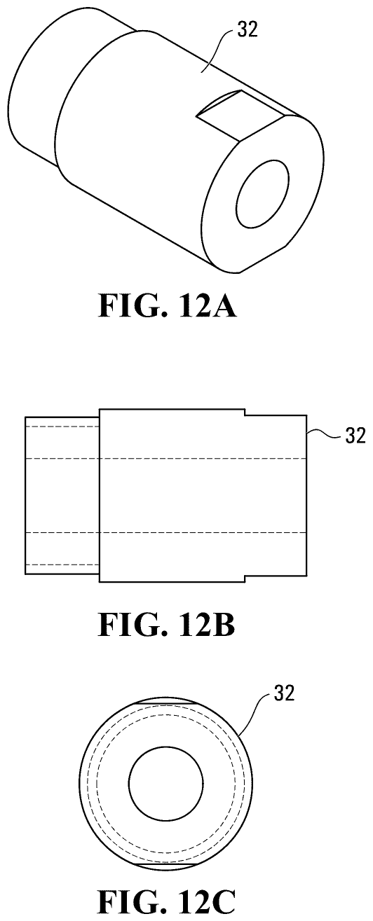

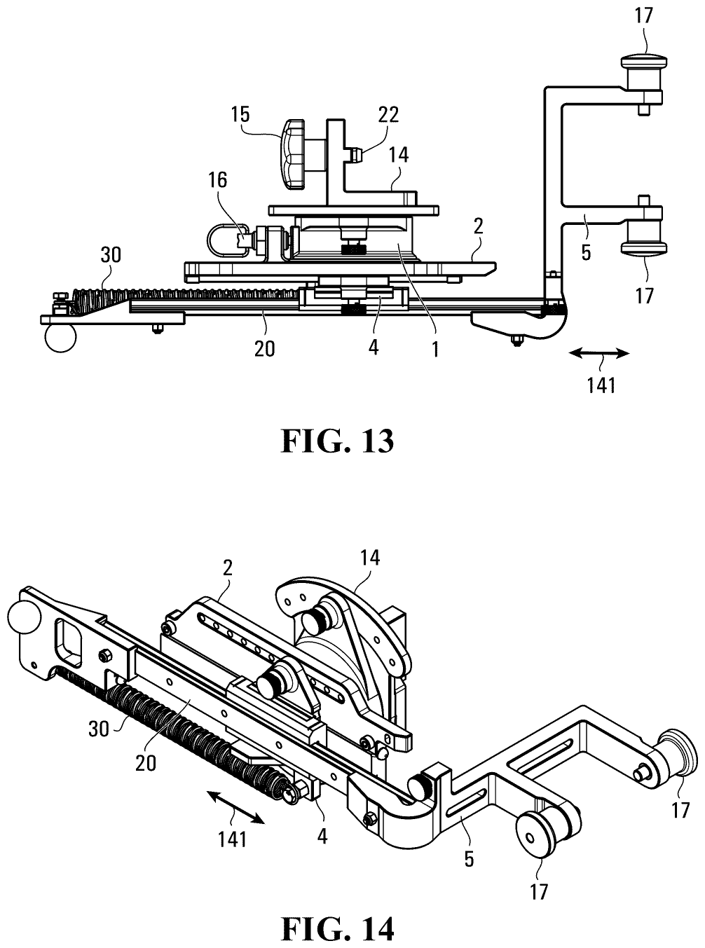

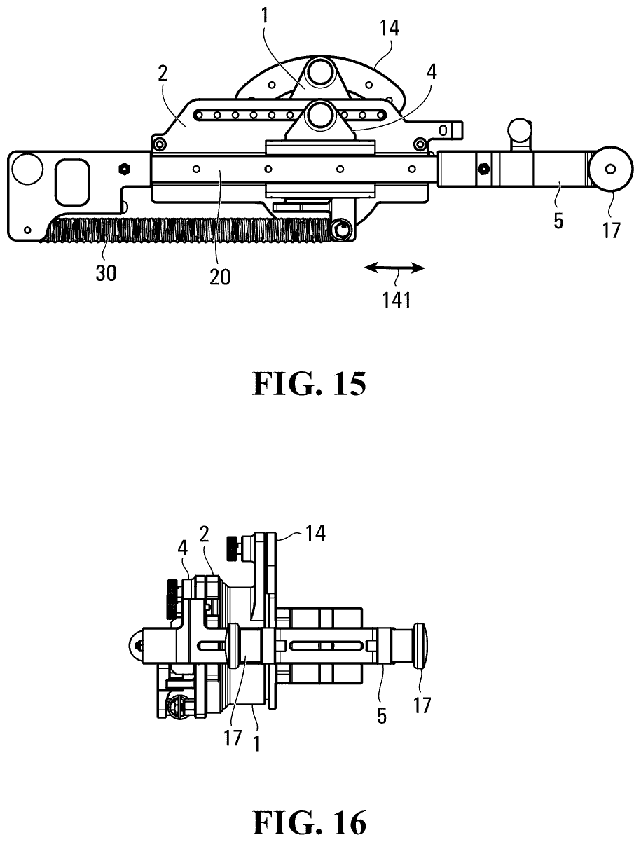

FIG. 4 illustrates at least some parts of the sensor-support body 134, including a pivot body (or pivot) 1, a slider body (or dovetail slider) 2, a retractable spring plunger 3, a guide plate 4, a clevis (or probe clevis or sensor clevis) 5, a needle-roller thrust bearing 6, a thrust washer 7, a dry-running thrust bearing 8, a sleeve bearing 9, a plastic knob 10, a torsion spring 11, a push-in bumper 12, a socket-head screw 13, a mounting body (or probe mount or sensor mount) 14, a knob 15, a cam-action indexing plunger 16, indexing plungers (or mini indexing plungers) 17, a flat-head screw 18, a ball-bearing carriage 19, a guide rail 20, a spring hook 21, a t-slot stud (or a drop-in t-slot stud) 22, a hex-screw head 23, a hex standoff 24, a hex standoff 25, a socket-head screw 26, a nylon-insert locknut 27, a socket-head screw 28, a socket-head screw 29, an extension spring 30, a shoulder screw 31, and a spring shaft 32. FIGS. 5A and 5B include details of those parts. FIGS. 6A-6G, 7A-7J, 8A-8G, 9A-9E, 10A-10E, 11A-11D, and 12A-12C include further details of parts of the sensor-support body 134, and FIGS. 13-16 illustrate other views of the sensor-support body 134.

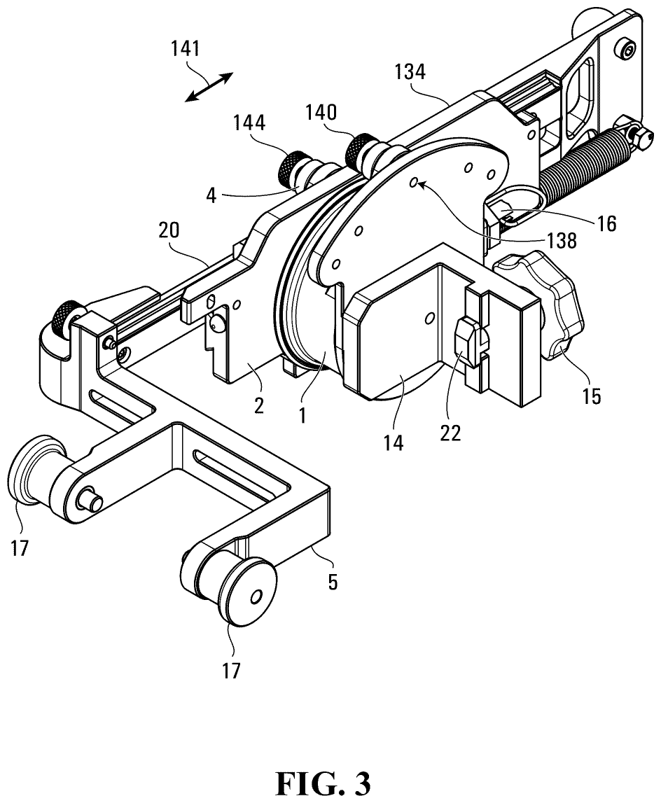

Referring back to FIGS. 3 and 4, the sensor-support body 134 includes the t-slot stud 22 and the knob 15 that may be turned to apply a force on the t-slot stud 22. When the t-slot stud 22 is received in a channel of the first elongate portion 104, as shown in FIG. 1 for example, turning the knob 15 may fasten the mounting body 14 of the sensor-support body 134, and thus the sensor-support body 134, to the first elongate portion 104 and thus to the support body 102 at one of many possible different adjustable positions along the first elongate portion 104. As a result, a position of the sensor-support body 134, relative to the support body 102, is adjustable in a direction 136 different from the direction 132 and along the first elongate portion 104. In the embodiment shown, the wheel 116 is generally cylindrical, and the direction 136 is an axial or longitudinal direction generally along an axis of rotation of the wheel 116, or a generally vertical direction in the orientation of FIGS. 1 and 2. Herein, an “axial” or a “longitudinal” direction is not limited to a geometrically precise axial or longitudinal direction, but rather may include directions that are substantially similar to a axial or longitudinal direction. Further, alternative embodiments may differ in other ways.

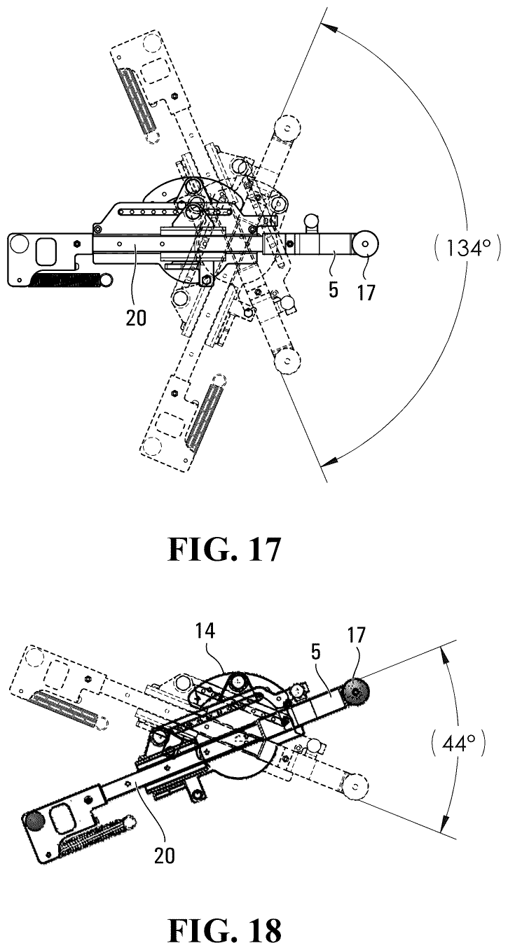

The sensor-support body 134 also includes the pivot body 1, which is pivotable (or, more generally, movable) relative to the mounting body 14. The mounting body 14 defines recesses or holes such as a recess or hole shown generally at 138, and a body 140 (such as a pin, which may be spring-loaded) may be received in one such recess or hole to fix a rotational position of the pivot body 1 relative to the mounting body 14 in one of a plurality of discrete positions defined by the recesses or holes such as the recess or hole 138. Alternative embodiments may differ, and a rotational position of the pivot body 1 relative to the mounting body 14 may be fixed in other ways. Also, in alternative embodiments, the pivot body 1 may be movable relative to the mounting body 14 in other ways that may not necessarily be pivotable or rotatable. FIG. 17 illustrates rotation of the pivot body 1 relative to the mounting body 14, which may be referred to as indexing up or down.

The torsion spring 11 is received in the pivot body 1, and the slider body 2 is rotatable relative to the pivot body 1 under resistance from the torsion spring 11. As a result, absent external torque, the torsion spring 11 urges the slider body 2 to a default rotational position relative to the pivot body 1, and external torque may rotate the slider body 2 resiliently to other rotational positions relative to the pivot body 1 and away from the default rotational position under resistance (or a resilient force) from the torsion spring 11. Herein, reference to urging or exerting a force does not require urging or exerting a force directly, but may include urging or exerting a force indirectly. For example, the torsion spring 11 may exert a force on, or urge, the slider body 2, which does not require direct contact between the torsion spring 11 and the slider body 2, but which rather may involve indirect interaction involving one or more intermediate parts that may transfer forces between the torsion spring 11 and the slider body 2.

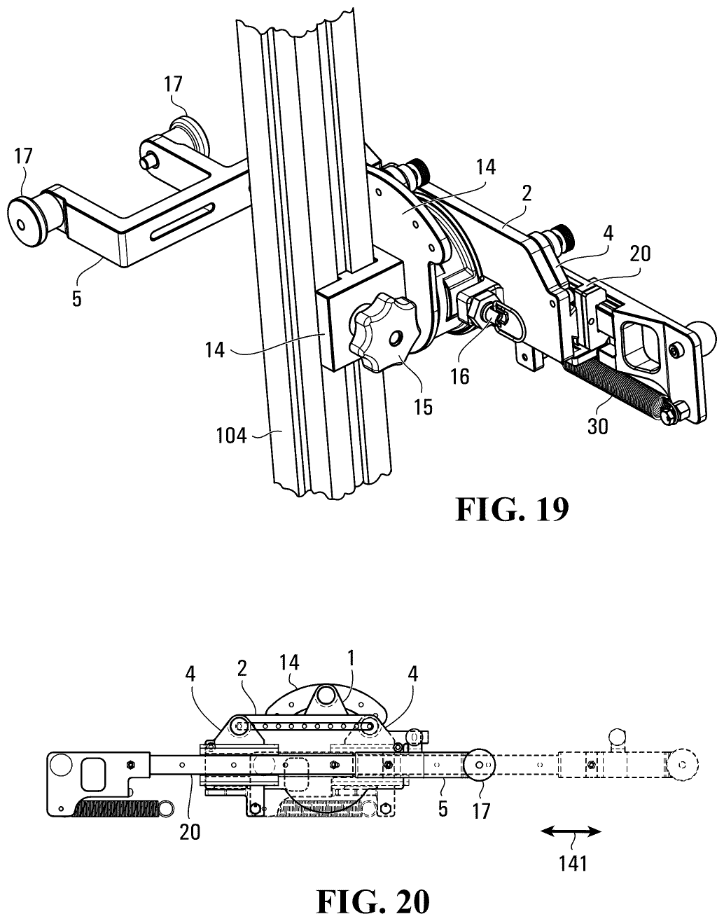

FIG. 18 illustrates rotation of the slider body 2 relative to the pivot body 1, which may be referred to as flexing up or down. FIGS. 3, 4, and 19 illustrate the cam-action indexing plunger 16 that may be positioned to prevent rotation of the slider body 2 relative to the pivot body 1 in one of one or more possible different adjustable positions. The torsion spring 11 is an example of a resilient body. Alternative embodiments may include one or more other resilient bodies or may omit the torsion spring 11. Further, in alternative embodiments, the slider body 2 may be movable relative to the pivot body 1 in other ways that may not necessarily be pivotable or rotatable.

The guide plate 4 is slidable relative to the slider body 2, and the guide rail 20 is slidably coupled to the guide plate 4. FIG. 20 illustrates linear movement (which may be referred to as linear travel) of the guide plate 4 and of the guide rail 20 relative to the slider body 2 in a linear direction 141.

The slider body 2 defines recesses or holes such as the recess or hole shown generally at 142, and a body 144 (such as a pin, which may be spring-loaded) may be received in one such recess or hole to fix a position of the guide plate 4 relative to the slider body 2. Alternative embodiments may differ, and a position of the guide plate 4 relative to the slider body 2 may be fixed in other ways. Further, in alternative embodiments, the guide plate 4 may be movable relative to the slider body 2 in other ways that may not necessarily be linear. Also, in alternative embodiments, the guide rail 20 may be movable relative to the guide plate 4 in other ways that may not necessarily be linear.

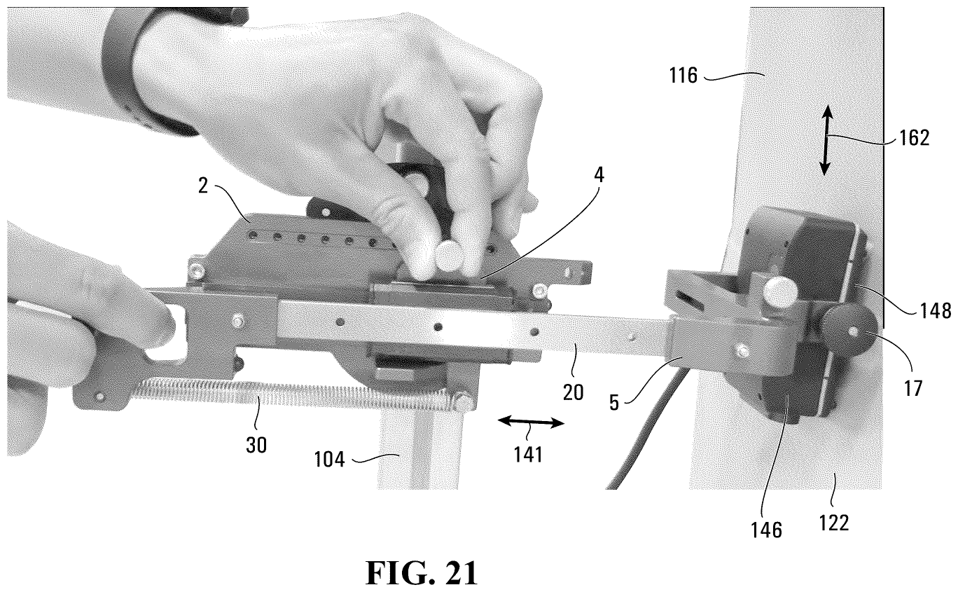

The extension spring 30 is attached at one end (or at one portion) to the guide rail 20 and at the other end (or at another portion) to the guide plate 4, and the extension spring 30 may exert a force on the guide rail 20 in the direction 141. As a result, sliding the guide plate 4 relative to the slider body 2 may adjust a location, relative to the support body 102, where a first portion of the extension spring 30 is attached to the support body 102, which may expand or contract the extension spring 30, which may vary a resilient force applied by the sensor-support body 134, and thus by the support body 102, on the guide rail 20 in the direction 141. FIG. 21 illustrates sliding the guide plate 4 relative to the slider body 2 to expand or contract the extension spring 30. The guide plate 4 may therefore be referred to as a “resilient-body-attachment body”.

The extension spring 30 is an example of a resilient body. Alternative embodiments may include one or more other resilient bodies or may omit the extension spring 30.

The clevis 5 is coupled to the guide rail 20 and configured to support one or more sensors or one or more probes on the guide rail 20. For example, in the embodiment shown, the indexing plungers 17 on the clevis 5 may retain a sensor or a probe such that the sensor-support body 134 supports the sensor or probe. The sensor-support body 134 and the support body 102 are therefore configured to support at least one sensor such that moving the support body 102 relative to the wheel 116 in the direction 132 causes the at least one sensor to move relative to the wheel 116 in the direction 132.

As indicated above, the torsion spring 11 urges the slider body 2 to a default rotational position relative to the pivot body 1. When the clevis 5 supports one or more sensors, the torsion spring 11 also urges the one or more sensors to a default rotational position relative to the pivot body 1. As also indicated above, the pivot body 1 is rotatable relative to the mounting body 14 and thus relative to the support body 102. Rotation of the pivot body 1 relative to the mounting body 14 adjusts the default rotational position of the slider body 2, and thus the default rotational position of one or more sensors supported by the clevis 5 and thus by the sensor-support body 134, relative to the mounting body 14 and thus relative to the support body 102. The default rotational position of the slider body 2 (and of one or more sensors supported by the sensor-support body 134) is thus adjustable relative to the support body 102. Further, because the body 140 may be received in one recess or hole (such as the recess or hole 138) of a plurality of recesses or holes, the default rotational position of the slider body 2 (and of one or more sensors supported by the sensor-support body 134) is thus adjustable relative to the support body 102 in one of a plurality of discrete positions defined by the recesses or holes such as the recess or hole 138.

The sensor-support body 134 is an example only, and alternative embodiments may differ. For example, alternative embodiments may omit one or more of the components of the sensor-support body 134 as described above, may include one or more alternatives to one, more than one, or all of the components of the sensor-support body 134 as described above, or may include one or more other components. Further, alternative embodiments may permit movement of one or more sensors or one or more probes in different directions or in different ways.

Examples of operation of the system 100 are shown in FIGS. 21-25. In general, turning the knob 15 may release the sensor-support body 134 from, and attach the sensor-support body 134 to, the first elongate portion 104 (and thus the support body 102) at one of many possible different adjustable positions along the first elongate portion 104. Further, the guide plate 4 and the guide rail 20 may be moved relative to the slider body 2 in the direction 141, which may be different from the directions 132 and 136, and which may be towards and away from the support body 102. In the embodiment shown, the wheel 116 is generally cylindrical, and the direction 141 is radial or generally radial relative to the wheel 116. However, alternative embodiments may differ.

As shown in FIG. 21, a sensor (or probe) 146 may be attached to the clevis 5, for example using one or both of the indexing plungers 17. The sensor 146 is thus supported by the sensor-support body 134 and by the support body 102. The sensor 146 has a generally planar face 148, and the sensor-support body 134 may be positioned such that the face 148 is positionable against a weld seam of the wheel 116. When the face 148 is positioned against the outer surface 122, the sensor 146 is able to detect defects in the wheel 116, for example using an eddy-current probe array of the sensor 146. The weld seam is an example only, and other portions of an object may be inspected. Further, although the illustrated examples involve inspection of the outer surface 122, alternative embodiments may include inspection of other surfaces, such as the inner surface 130 or another surface.

FIG. 21 also illustrates movement of the guide plate 4 relative to the slider body 2 to adjust tension in the spring 30.

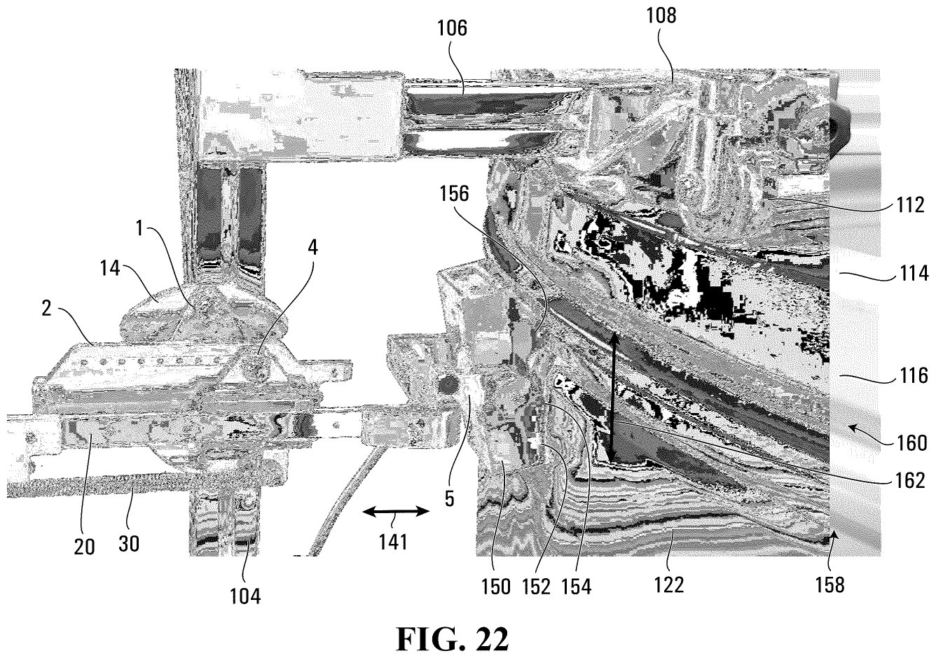

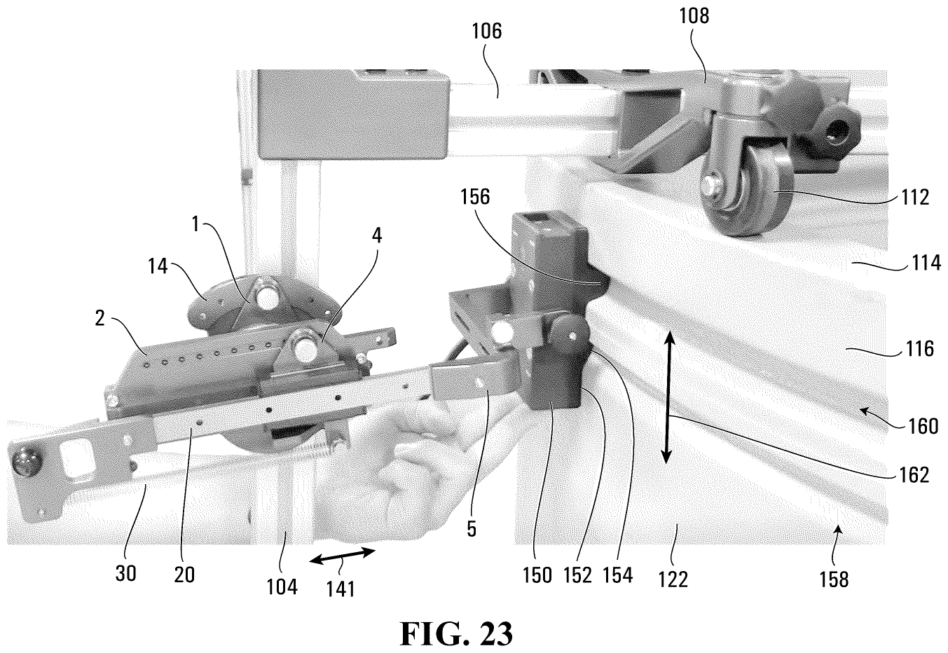

FIG. 22 illustrates a sensor (or probe) 150 having a face 152 defining projections 154 and 156 sized to be received in grooves 158 and 160 respectively of the wheel 116. The sensor 150 may be attached to the clevis 5, for example using one or both of the indexing plungers 17, and may thus supported by the sensor-support body 134 and by the support body 102. In FIG. 22, the sensor 150 is positioned such that the projections 154 and 156 are not received in the grooves 158 and 160 respectively. However, FIG. 23 illustrates movement of the sensor 150 relative to the support body 102 in a direction (or second direction) 162 different from the directions 132 and 141 and along the outer surface 122 to position the projections 154 and 156 in the grooves 158 and 160 respectively. Such movement of the sensor 150 relative to the support body 102 may be accommodated by rotation of the slider body 2 relative to the pivot body 1 as described above, and by linear movement of the guide rail 20 relative to the slider body 2. The sensor-support body 134 therefore permits movement of the sensor 150 relative to the support body 102 in the direction 162 by rotation of the slider body 2 (or, more generally, by rotation of at least a portion of sensor-support body 134) relative to the support body 102, although alternative embodiments may differ. The spring 30 may maintain a resilient force on the guide rail 20 and thus on the sensor 150 in the direction 141, thereby resiliently urging the sensor 150 against the outer surface 122 throughout some or all of such movement of the sensor 150 relative to the support body 102. The resilient force exerted by the spring 30 on the sensor 150 and in the direction 141 may be generally constant throughout some or all of movement of the sensor 150 relative to the support body 102. Movement of the guide plate 4, of the guide rail 20, or both, relative to the slider body 2 in the direction 141, permits movement of the sensor 150 relative to the support body 102 in the direction (or a third direction) 141.

In some embodiments, the direction 162 may be the same as or generally the same as the direction 136. In the embodiment shown, the wheel 116 is generally cylindrical, and the direction 162 is an axial or longitudinal direction generally along an axis of rotation of the wheel 116, or a generally vertical direction in the orientation of FIGS. 1 and 2. Again, an “axial” or a “longitudinal” direction herein is not limited to a geometrically precise axial or longitudinal direction, but rather may include directions that are substantially similar to an axial or longitudinal direction. Further, alternative embodiments may differ in other ways.

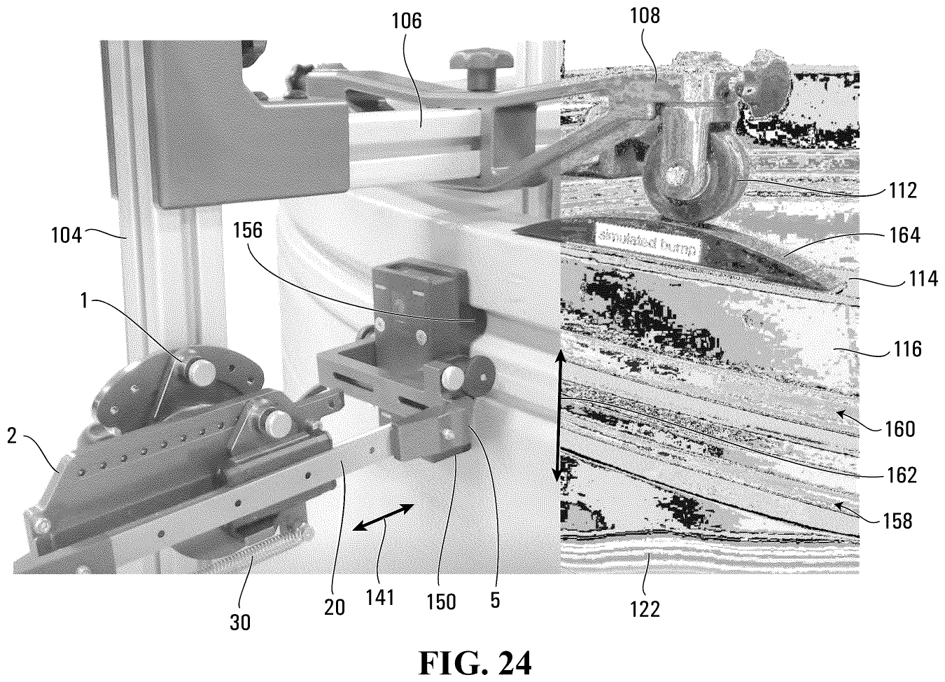

As shown in FIG. 24, the inner rim 114 may be uneven, and may for example include a bump 164. When the slidable support body 108 passes over the bump 164, the entire support body 102 may rise relative to the wheel 116. However, the projections 154 and 156 may remain in the grooves 158 and 160 respectively, so movement of the entire support body 102 relative to the wheel 116, in a direction other than the direction 132 around the wheel 116, may also cause movement of the sensor 150 relative to the support body 102 in the direction 162. Again, such movement of the sensor 150 relative to the support body 102 may be accommodated by rotation of the slider body 2 relative to the pivot body 1 as described above, and by linear movement of the guide rail 20 relative to the slider body 2, and the spring 30 may maintain a resilient force on the guide rail 20 and thus on the sensor 150 in the direction 141, thereby resiliently urging the sensor 150 against the outer surface 122 throughout some or all of such movement of the sensor 150 relative to the support body 102.

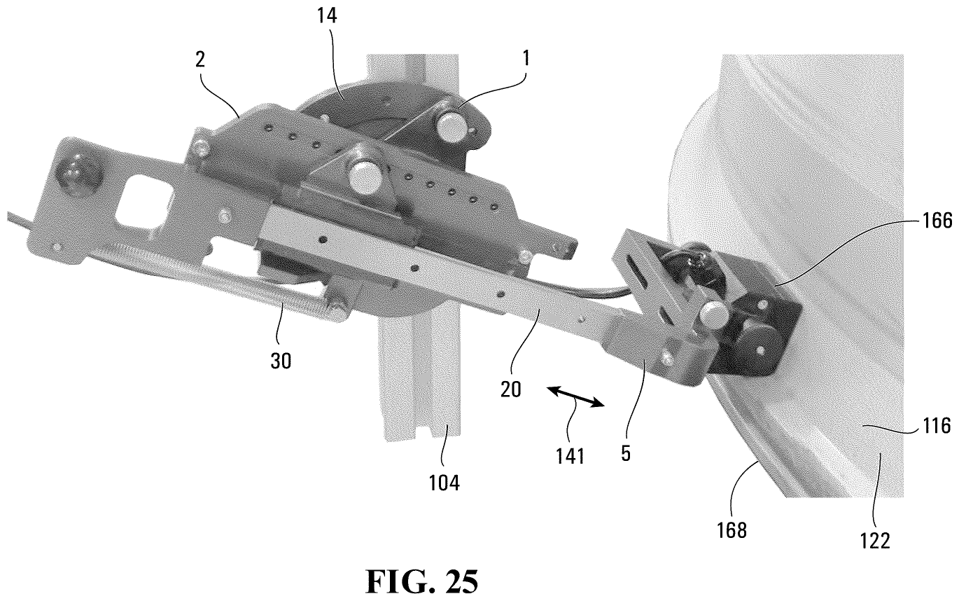

FIG. 25 illustrates rotation of the pivot body 1 relative to the mounting body 14 to position a sensor 166, supported by the clevis 5 as described above, against the outer surface 122 near an outer rim 168 opposite the inner rim 114. The sensor 166 is thus supported by the sensor-support body 134 and by the support body 102.

Sensors such as the sensor 146, 150, or 166 may include eddy-current arrays or any other sensors that may be appropriate for an object to be inspected.

With the sensor 146, 150, or 166 supported as shown in FIGS. 21 and 23-25, for example, the support body 102 may be moved relative to the wheel 116 in the direction 132, which may cause such sensor 146, 150, or 166 to move relative to the wheel 116 in the direction 132 to inspect the wheel. During some of all of such movement of the support body 102 relative to the wheel 116 in the direction 132, or at other times, the sensor 146, 150, or 166 may move in the direction 162 relative to the support body 102. Such movement of the sensor 146, 150, or 166 in the direction 162 relative to the support body 102 may facilitate inspection of particular locations, such as allowing the sensor 150 to be aligned with the grooves 160 and 162 as in FIGS. 22 and 23, or allowing the sensor 166 to be aligned with the outer surface 122 near the outer rim 168 as in FIG. 25. Further, such movement of the sensor 146, 150, or 166 in the direction 162 relative to the support body 102 may accommodate uneven surfaces such as the bump 164 in FIG. 24. In general, during such inspection, the sensor 146, 150, or 166 may be in communication with the computing device 133 and may transmit one or more signals to the computing device 133, which may accumulate data from such a sensor, store data from such a sensor, display data from such a sensor, analyze data from such a sensor, or a combination of two or more thereof.

Herein, references to “first”, “second”, and “third” are for clarity only, do not imply any sequence or significance of parts, and do not imply any necessary existence of other parts. For example, references to “first” and “second” parts do not imply any sequence or significance of those parts. As another example, reference to a “first”, to a “second”, or to a “third” part does not imply any necessary existence of other parts.

Although specific embodiments have been described and illustrated, such embodiments should be considered illustrative only and not as limiting the invention as construed according to the accompanying claims.

1. A method of inspecting an object, the method comprising:
moving a support body relative to the object in a first direction along a surface of the object;
wherein the support body supports at least one sensor such that moving the support body relative to the object in the first direction causes the at least one sensor to move relative to the object in the first direction;
wherein the at least one sensor is movable relative to the support body in a second direction along the surface of the object and different from the first direction during at least a portion of time while moving the support body relative to the object in the first direction; and
wherein the at least one sensor is movable relative to the support body in a third direction different from the first and second directions and towards and away from the support body.

2. The method of claim 1 wherein, during at least a portion of time while moving the support body relative to the object in the first direction, the support body is supported by the object.

3. (canceled)

4. The method of claim 1, wherein a sensor-support body supports the at least one sensor relative to the support body.

5. The method of claim 4 wherein the sensor-support body permits movement of the at least one sensor relative to the support body in the second direction.

6. The method of claim 5 wherein at least a portion of the sensor-support body is rotatable relative to the support body, and the sensor-support body permits the movement of the at least one sensor relative to the support body in the second direction by rotation of the at least a portion of the sensor-support body relative to the support body.

7. The method of claim 4, wherein the sensor-support body permits movement of the at least one sensor relative to the support body in the third direction.

8-10. (canceled)

11. The method of claim 1 further comprising adjusting a resilient force urged by a first at least one resilient body on the at least one sensor, the first at least one resilient body resiliently urging the at least one sensor against the surface of the object.

12. The method of claim 11 wherein adjusting the resilient force urged by the first at least one resilient body on the at least one sensor comprises adjusting a location, relative to the support body, where a first portion of the first at least one resilient body is attached to the support body, wherein a second portion of the first at least one resilient body exerts the resilient force on the at least one sensor.

13. (canceled)

14. The method of claim 1 wherein movement of the at least one sensor relative to the support body in the second direction is against a resilient force from a second at least one resilient body.

15. The method of claim 14 wherein the second at least one resilient body urges the at least one sensor in the second direction towards an adjustable default position of the at least one sensor relative to the support body.

16-31. (canceled)

32. An apparatus for inspecting an object, the apparatus comprising:
a support body movable relative to the object in a first direction along a surface of the object;
wherein the support body is configured to support at least one sensor such that moving the support body relative to the object in the first direction causes the at least one sensor to move relative to the object in the first direction, such that the at least one sensor is movable relative to the support body in a second direction along the surface of the object and different from the first direction while moving the support body relative to the object in the first direction, and such that the at least one sensor is movable relative to the support body in a third direction different from the first and second directions and towards and away from the support body.

33. The apparatus of claim 32 wherein the support body defines a space for receiving at least a portion of the object, the support body comprising at least one support surface facing the space to support the support body on the object when the space receives the at least a portion of the object.

34. The apparatus of claim 32 further comprising a sensor-support body configured to support the at least one sensor relative to the support body.

35. The apparatus of claim 34 wherein the sensor-support body permits movement of the at least one sensor relative to the support body in the second direction.

36. The apparatus of claim 35 wherein at least a portion of the sensor-support body is rotatable relative to the support body, and the sensor-support body permits the movement of the at least one sensor relative to the support body in the second direction by rotation of the at least a portion of the sensor-support body relative to the support body.

37. The apparatus of claim 34, wherein the sensor-support body permits movement of the at least one sensor relative to the support body in the third direction.

38-40. (canceled)

41. The apparatus of claim 32 further comprising a first at least one resilient body configured to urge resiliently the at least one sensor against the surface of the object, wherein a resilient force urged by the first at least one resilient body on the at least one sensor is adjustable.

42. (canceled)

43. (canceled)

44. The apparatus of claim 32 further comprising a second at least one resilient body configured to impose a resilient force against movement of the at least one sensor relative to the support body in the second direction.

45. The apparatus of claim 44 wherein the second at least one resilient body is configured to urge the at least one sensor in the second direction towards an adjustable default position of the at least one sensor relative to the support body.

46-50. (canceled)

51. The apparatus of claim 32 wherein the support body is configured to be supported by the object such that the support body is movable relative to the object in the first direction.

52-65. (canceled)
